(19) United States
(12) Patent Application Publication
(10) Pub. No.: US 2023/0245785 A1
(43) Pub. Date: Aug. 3, 2023

(54) METHOD FOR CONSTRUCTING FUNCTIONAL CLASSIFIERS FOR MICROBIOME ANALYSIS

(71) Applicant: International Business Machines Corporation, Armonk, NY (US)

(72) Inventors: Edward E. Seabolt, Georgetown, TX (US); James H. Kaufman, San Jose, CA (US)

(21) Appl. No.: 17/590,597

(22) Filed: Feb. 1, 2022

(51) Int. Cl. G16H 50/70; G06F 16/28; G06F 16/22

(52) U.S. Cl. CPC G16H 50/70; G06F 16/285; G06F 16/2246

(57) ABSTRACT

A method for classifying microbial function within any microbiome can be carried out with any coding system. The method, which does not entail measuring the distance between sequences, includes: (1) selecting a reference database that links a coding system to a set of biological sequences; (2) constructing an N×M matrix with each row (N) representing a code from the coding system, each column (M) representing a single biological sequence from the set, and cells representing the presence, absence, or frequency of the single biological sequence for one or more codes; (3) computing the pair-wise distance between the rows of the matrix to form an N×N matrix, wherein N is the number of codes in the matrix; (4) clustering the results to form a data tree; (5) generating a taxonomic tree from the cluster results; and (6) applying a classification tool to the taxonomic tree to classify the microbiome.

### TECHNICAL FIELD

The present invention relates generally to classification of biological data, and more specifically to a method of classifying microbiome data in human organisms using any system for functional coding of biological data.

### BACKGROUND OF THE INVENTION

Through the application of metagenetics and sequence technologies, information relating to the human microbiome has shown an association between microbiome imbalances, certain physiological conditions and/or diseases. Gaining an understanding of the different microorganisms that exist within the human microbiome across different physiological conditions and disease states is a first step in the development of targeted treatments and therapies. Such an understanding has thus far been statistically difficult to achieve due to the large number of taxa existing within collected samples.

Supervised learning artificial intelligence (AI) has been used to classify large numbers of biological information. Supervised learning uses trained and labeled samples to make predictions for new unlabeled samples. The large number of taxa within the human microbiome makes supervised learning difficult since the large number of unknown features within the human microbiome far exceeds the small number of known observations. The result is that an AI system cannot be properly trained for microbiome classification.

### SUMMARY OF THE INVENTION

In one aspect, the present invention relates to a method of constructing a microbiome classifier comprising: selecting a reference database comprising a set of biological sequences, wherein each biological sequence is annotated with a code from at least one coding system; constructing a matrix comprising rows, columns, and cells, wherein each row represents one code from the at least one coding system, each column represents a single biological sequence from the set, and the cells show the presence, absence, or frequency of the single biological sequences for one or more codes of the at least one coding system; computing pair-wise distance between the rows of the matrix to arrive at a single pair-wise distance value for each code in the set, wherein the pair-wise distance computations between all of the rows of the matrix is an N×N matrix, wherein N is the number of codes in the matrix; clustering the pair-wise distance values for each biological sequence to form a data structure tree comprising clusters, wherein the clusters represent a relationship between a code of the at least one coding system and one or more biological sequences; constructing a taxonomic tree comprising internal nodes and leaf nodes wherein the internal nodes represent the clusters of the data structure tree and both the internal nodes and the leaf nodes represent the biological sequences; and applying a classification tool to the final taxonomic tree to classify a microbiome comprised of the biological sequences.

In another aspect, the present invention relates to a method of constructing a microbiome classifier comprising: selecting a reference database comprising a set of protein domain sequences, wherein each protein domain sequence is annotated with an a code from at least one coding system; constructing a matrix comprising rows, columns, and cells, wherein each row represents one code from the at least one coding system, each column represents a single protein domain sequence from the set, and the cells show the presence, absence, or frequency of the single protein domain sequences for one or more codes of the at least one coding system; computing pair-wise distance between the rows of the matrix to arrive at a single pair-wise distance value for each code in the set, wherein the pair-wise distance computations between all of the rows of the matrix is an N×N matrix, wherein N is the number of codes in the matrix; clustering the pair-wise distance values for each protein domain sequence to form a data structure tree comprising clusters, wherein the clusters represent a relationship between one or more codes of the at least one coding system and one or more protein domain sequences; constructing a taxonomic tree comprising internal nodes and leaf nodes wherein the internal nodes represent the clusters of the data structure tree and both the internal nodes and the leaf nodes represent the protein domain sequences; and applying a classification tool to the final taxonomic tree to classify a microbiome comprised of the protein domain sequences.

Additional aspects and/or embodiments of the invention will be provided, without limitation, in the detailed description of the invention that is set forth below.

### BRIEF DESCRIPTION OF THE DRAWINGS

FIG. 1 is a schematic diagram showing the microbiome functional classifier construction steps.

FIG. 2 is a schematic diagram showing the functional taxonomy of tree construction.

FIG. 3 is a schematic diagram showing how performance is quantitatively measured using a combination of raw sequence reads and annotated ground truth from whole genomes.

FIG. 4 is an AUC:ROC graph showing the accuracy of the functional classifier to classify the bacterial species Escherichia coli.

FIG. 5 is an AUC:ROC graph showing the accuracy of the functional classifier to classify the bacterial species Listeria monocytogenes.

FIG. 6 is an AUC:ROC graph showing the accuracy of the functional classifier to classify the bacterial species Salmonella enterica.

FIG. 7 is an AUC:ROC graph showing the accuracy of the functional classifier to classify the bacterial species Staphylococcus aureus.

FIG. 8 is an AUC:ROC graph showing the accuracy of the functional classifier to classify the Betacoronavirus RNA virus.

### DETAILED DESCRIPTION OF THE INVENTION

Set forth below is a description of what are currently believed to be preferred aspects and/or embodiments of the claimed invention. Any alternates or modifications in function, purpose, or structure are intended to be covered by the appended claims. As used in this specification and the appended claims, the singular forms “a,” “an,” and “the” include plural referents unless the context clearly dictates otherwise. The terms “comprise,” “comprised,” “comprises,” and/or “comprising,” as used in the specification and appended claims, specify the presence of the expressly recited components, elements, features, and/or steps, but do not preclude the presence or addition of one or more other components, elements, features, and/or steps.

As used herein, the term “taxonomy” refers to the hierarchical classification of biological sequences according to groups based upon the microbial function of the biological sequences within a microbiome. The hierarchical classification is generally in the graphical form of a tree (by convention, drawn growing downwards) comprised of a collection of nodes where each node is a data structure having a value. The nodes of the tree may be internal or external, the latter also known as leaf nodes. The topmost node of a tree is called the root node, which is the node at which algorithms on the tree begin. All nodes branching from the parent are child nodes and each child node has at least one parent node. An internal node is any node of a tree that has child nodes and a leaf node is a node that does not have any child nodes.

As used herein, the term “microbiome” refers to a community of microorganisms that live together within a given habitat. The living members of a microbiome are referred to as microbiota and include, without limitation, bacteria, archaea, fungi, algae, small protists, phages, viruses, plasmids, and mobile genetic elements (MGEs). Examples of MGEs include, without limitation, segments of DNA that encode enzymes and other proteins that mediate the movement of DNA within genomes (intracellular mobility) or between bacterial cells (intercellular mobility).

As used herein, the term “microbial function” refers to the activity of microorganisms within human cells. Examples of microbial functions include, without limitation, digestion, vitamin production (e.g., B, B12, thiamin, riboflavin, K), protection against bacteria that cause disease, development of the immune system, and detoxifying harmful chemicals.

As used herein, the terms “biological sequence(s)” and “sequence(s)” refer to gene sequences comprised of nucleic acids (i.e., a nucleotide sequence) and/or protein sequences comprised of amino acids (i.e., an amino acid sequence). The biological sequences may be in the form of a single, continuous molecule of nucleic acids or amino acids, a physical or genetic map, or a composite data structure. Within biological sequences are motifs and domains (also referred to herein as “domain sequences”). A “motif” is a short, conserved sequence pattern associated with distinct functions of a nucleic acid or a protein. A motif is often associated with a distinct structural site preforming a particular function. For example, a typical motif is a zinc-finger motif, which is 10-20 amino acids long. A “domain” is a conserved sequence pattern that is an independent functional and structural unit. A domain is generally longer than a motif with domains ranging from 40-700 residues (nucleic acids or amino acids) with 100 residues being an average length. Motifs and domains are evolutionarily more conserved than other regions of a gene sequence or protein sequence and tend to evolve as units, which are gained, lost, or shuffled as one module. Domains that show sequence similarity and/or related functions are grouped into families and domains having common ancestry are grouped into superfamilies.

As used herein, the term “whole genome sequencing” and “WGS” refer to the construction of the complete nucleotide and/or amino acid sequence of a genome.

As used herein, the term “pair-end reads” refers to the two ends of the same DNA molecule. With a pair-end read, a DNA molecule is sequenced towards one end and turned around for sequencing to the other end; the two sequences are the pair-end reads. Unlike a gene, which is a nucleic acid sequence that has been identified through a genomic annotation process, a pair-end read represents unassembled DNA that is sequenced.

As used herein, the term “pair-wise distance” is a data reduction method by which many different numerical values are reduced to a single number. Generally, the term pair-wise distance refers to the results of a calculation where all pairs of a sequence are evaluated and the differences between all of the pairs of the sequence are transformed into a single number representing a distance. The pairs of the sequence may be between two horizontal, two vertical, and/or two diagonal pairs within the rows and columns of a matrix.

As used herein, the term “cosine similarity” refers to a measure of similarity between two non-zero vectors of an inner product space. Cosine similarity is equal to the cosine of the angle between the two non-zero vectors, but not their magnitude. The cosine similarity is bounded by the interval [−1,1] for any angle θ. For example, two vectors with the same orientation have a cosine similarity of 1 while two vectors oriented at right angles relative to each other have a similarity of 0, and two vectors diametrically opposed have a similarity of −1. Unit vectors are maximally similar when they are parallel and maximally dissimilar when they are orthogonal (perpendicular). Cosine similarity is particularly useful in positive spaces where the outcome is bounded in [0,1].

As used herein, the term “Euclidean distance” refers to a formula that is used to find the distance between two points on a plane. The Euclidean distance is calculated from the Cartesian coordinates of the points on the plane using the Pythagorean formula. For example, for the distance between two points, (x1 1, y1 1) and (x2 2, y2 2), a Euclidean distance can be calculated according to Formula (1):

d(x,y)=√[(x²−x¹)+(y₂−y₁)²] (1)

As used herein, the term “Hamming distance” refers to a string metric for measuring the edit distance between two sequences. Within the context of the present invention, 37 string’ is a biological sequence as defined herein and a “string metric” is a function that measures the distance (i.e., inverse similarity) between two strings and provides a number indicating an algorithm-specific indication of distance. An “edit distance” is a method of quantifying how dissimilar two strings are two one another by counting the number of operations required to transform one string to the other. By way of illustration, the Hamming distance between two equal length biological sequences (i.e., strings) is the number of biological sequence residues at which the two biological sequences are different.

As used herein, the term “Jaccard distance” refers to a measure of the dissimilarity between sample sets. It is complementary to the Jaccard coefficient, which measures the similarity between sample sets, and is obtained by subtracting the Jaccard coefficient from 1. The Jaccard distance is used to calculate an n n matrix for clustering and multi-dimensional scaling of n sample sets. The Jaccard distance may be calculated by dividing the difference of the sizes of the union and the intersection of the two sets by the size of the union according to Formulas (2) or by taking the ratio of the size of the symmetric distance to the union according to Formula (3).

dⱼ(A,B)=1−J(A,B)=|A∪B|−|A∩B/|A∪B| (2)

AΔB=(A∪B)−(AπB) (3)

As used herein, the term “sparse matrix” refers to a matrix in which most of the elements are zero (a matrix where most of the elements have non-zero values is considered to be a dense matrix). In a sparse matrix, the number of non-zero elements is roughly equal to the number of rows or columns and the matrix has few pair-wise interactions.

As used herein, the terms “cluster” and/or “clustering” refers to hierarchical clustering where similar sequences are closer together than different sequences. Within the context of the present invention, the clustering of sequences forms the initial taxonomic tree (also referred to herein as a “data tree”) for the method of microbiome classification described herein.

As used herein, the term “unique” is meant to refer to a single occurrence of an element of the claimed method. For example, the terms “unique identifier” and “UID” refers to a label that is guaranteed to be unique among all identifiers for an object or for a specific purpose. Examples of UIDs include, without limitation, serial numbers, random numbers, and hash functions. A hash function is a computer program that takes a data input of arbitrary length and outputs a UID of fixed length. Within the context of biological data, hashing can be used on data as small as a codon and as large as an entire genome. The length of the output or hash is dependent on the hashing algorithm. Most hashing algorithms have a hash length between 160-512 bits. Examples of hashing algorithms include, without limitation, MD5 (Message Direct Algorithm, version 5), SHA-1 (Secure Hash Algorithm, original), SHA-2 (SHA suite of hashing algorithms including SHA-224, SHA-256, SHA-384, and SHA-512), LANMAN (Microsoft LAN Manager, Microsoft Corporation, Redmond, Wash., USA), and NTLM (NT LAN Manager, successor to LANMAN, Microsoft Corporation, Redmond, Wash., USA). The term “unique sequence” is meant to refer to a single occurrence of a sequence within the N×N matrix defined herein. It is to be understood that the unique sequence is an operation of mathematics and that more than one occurrence of the unique sequence may occur within the subject organism.

As used herein, the term “ROC” refers to a “receiver operating characteristic” curve, which is a graphical plot that illustrates the diagnostic ability of a binary classifier system where the discrimination threshold is varied. An ROC curve plots the true positive rate (TPR; sensitivity, recall, probability of detection) against the false positive rate (FPR; probability of false alarm, fall-out) at various threshold settings. For any binary classification system, the ROC curve thus plots sensitivity or recall as a function of fall-out.

As used herein, the term “AUC” refers to “area under the curve,” which provides the quantitative performance measurement for a binary classifier system. In an ROC curve, an AUC has a value between 0 and 1 where 0 represents chance performance, 0.5 represents an uninformative classifier, and 1 represents perfect performance.

Described herein is a method of classifying microbial function within any microbiome with any coding system. The construction of the microbiome functional classifier comprises:
(1) selecting a reference database comprising a set of biological sequences, wherein each biological sequence is annotated with a code from at least one coding system;
(2) constructing an N×M matrix comprising rows (N), columns (M), and cells, wherein each row represents one code from the at least one coding system, each column represents a single biological sequence from the set, and the cells show the presence, absence, or frequency (1/0, T/F) of the single biological sequences for one or more codes of the at least one coding system;
(3) computing pair-wise distance between the rows of the matrix to arrive at a single pair-wise distance value for each code in the set, wherein for each code, the end result is a N×N matrix where N is the number of codes (i.e., rows) in the matrix;
(4) clustering the pair-wise distance values for each biological sequence to form a data structure tree comprising clusters, wherein the clusters represent a relationship between a code of the at least one coding system and one or more biological sequences;
(5) constructing a taxonomic tree comprising internal nodes and leaf nodes wherein the internal nodes represent the clusters of the data structure tree and both the internal nodes and the leaf nodes represent the biological sequences; and
(6) applying a classification tool to the final taxonomic tree to classify a microbiome comprised of the biological sequences.

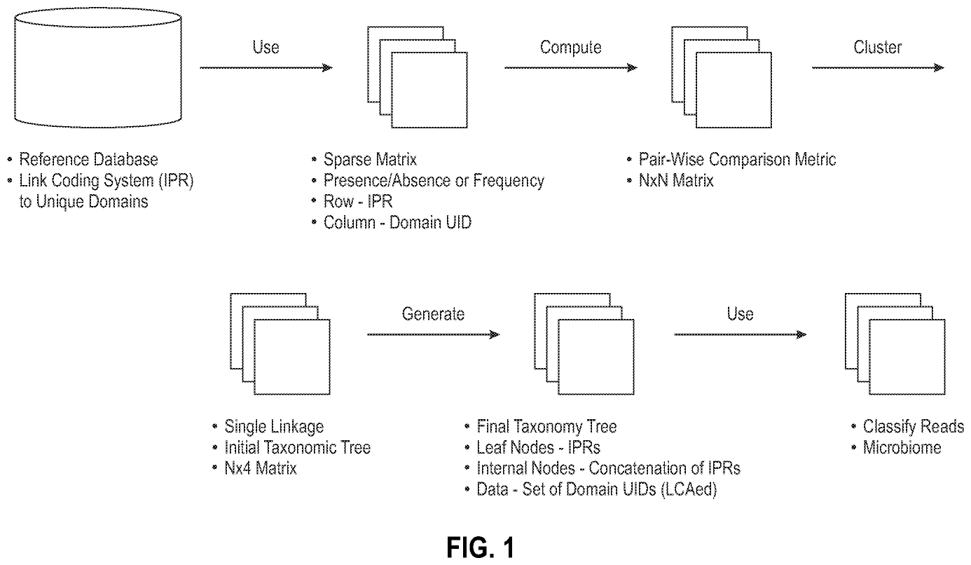

FIG. 1 shows application of the microbiome classification method to construct taxonomic trees that relate microbial function and/or phenotype to protein domain sequences.

The functional classifier is capable of using any coding system to classify a microbiome. Examples of coding systems that may be used for the functional classifier, include, without limitation, InterProScan (EMBL European Bioinformatics Institute, ebi.ac.uk/interpro/), KFGG/EC (Kyoto Encyclopedia of Genes and Genomics, kegg.jp; Enzyme Commission), and Gene Ontology (GO) (Open Biomedical Ontologies, OBO Foundry, obofoundry.org).

InterPro (IPR) is a database of protein families, domains, and functional sites in which identifiable features found in known proteins can be applied to new protein sequences in order to functionally characterize them. InterProScan is a software package that allows users to scan sequences against member database signatures and annotate proteins with functional IPR codes that exist in a hierarchy at the domain, family, and homologous superfamily levels. InterProScan coding is not organized as a tree. Consequently, InterProScan cannot be used alone for classifying microbiome samples; however, when InterProScan is integrated into the method described herein, the software is capable of successfully classifying microbiome data.

KEGG is a collection of databases directed to genomes, biological pathways, disease, drugs, and chemical substances and EC is a numerical classification scheme for enzymes based on the chemical reactions that they catalyze. KFGG/EC coding is organized as a tree. While KEGG/EG can be used independently to classify proteins, it can only classify ˜40% of the annotated protein domains.

GO is a bioinformatics initiative to unify the representation of gene and gene product attributes across all species. The GO initiative (1) maintains and develops a controlled vocabulary of gene and gene product attributes; (2) annotates gene and gene products and assimilates and disseminates the annotation data; and (3) provides tools for easy access to all aspects of the data provided by the project and enables functional interpretation of the data. GO annotations include a gene product identifier and generally include reference to a journal, a code denoting the type of evidence upon which the annotation is based, and the data and creator of the annotation.

Within the context of IPR coding, the functional classifier described herein is able to use 100% of the available domains that have associated IPR codes to build the taxonomy. Unlike currently used coding systems, the functional classifier does not measure sequence distances; instead, it operates by comparing individual domain sequences that are identified by the coding system with unique identifiers (UIDs). In this way, the functional classifier described herein is computationally efficient and therefore, is less expensive and resource intensive than currently used coding systems. The result is a classifier with a large set of domains as evidence, which may be used with any coding system that is directed to some function of biological sequences, including, without limitation, the KFGG/EC, InterProScan, and GO coding system referenced herein.

In one embodiment, the biological sequences that may be classified by the method include pair-end reads, gene sequences, protein sequences, and combinations thereof. In another embodiment, the biological sequences are annotated with one or more functional codes selected from (i) nucleic and/or amino acid pathways; (ii) chemical reactions involving nucleic acid and/or proteins; (iii) protein reactions initiated by enzymes; and (iv) hierarchical functional codes relating to domain, family, and homologous superfamily levels.

In a further embodiment, the biological sequence is a protein sequence and the coding system annotates the protein sequence with information relating to (i) enzymes that catalyze reactions with the protein sequence and/or (ii) reactions and/or pathways that the protein sequence undergoes. In another embodiment, the coding system defines a function and/or phenotype of a protein domain sequence.

In a further embodiment, the reference database is Functional Genomics Platform (FGP) (International Business Machines Corporation, Armonk, N.Y., USA). FGP is a relational database that organizes microbial organisms (genotype) and their associated protein domains according to their biological functions (phenotypes). In another embodiment, UniProt (Universal Protein resource, UniProt Consortium, accessible at uniprot.org) may be used to build a reference database. With UniProt, each sequence stored therein contains information on cross-reference databases that describe the sequence functions. In this way, a reference database may be built by using the information in UniProt.

In a further embodiment, each row of the matrix is a vectorization of the distinct features (e.g., domains) that are related to the code. In another embodiment, each biological sequence (which may be a protein domain sequence) in the columns of the matrix is coded with a single unique identifier (UID). In a further embodiment, the matrix is a sparse matrix and each row of the matrix is a sparse vectorization of the distinct features (e.g., domains) that are related to the code. In another embodiment, the metric used to compute the pair-wise distance between the row vectorizations (whether sparse or dense) is selected from the group consisting of cosine similarity, Euclidean distance, Hamming distance, Jaccard distance, and combinations thereof. The end result of the pair-wise distance computations between all of the rows of the matrix is an N×N matrix, wherein N is the number of codes in the matrix.

In a further embodiment, the clustering of the computational results is hierarchical as is the coding system. With hierarchical coding, new clusters with similar representations are formed via single linkages in a predefined top to bottom tree formation.

In another embodiment, the clustering of the computational results is hierarchical, but the coding system is non-hierarchical. With non-hierarchical coding, new clusters are formed by the merging or splitting of clusters without following a hierarchical tree formation. Non-hierarchical coding is useful for maximizing or minimizing evaluation criteria from the clustered data.

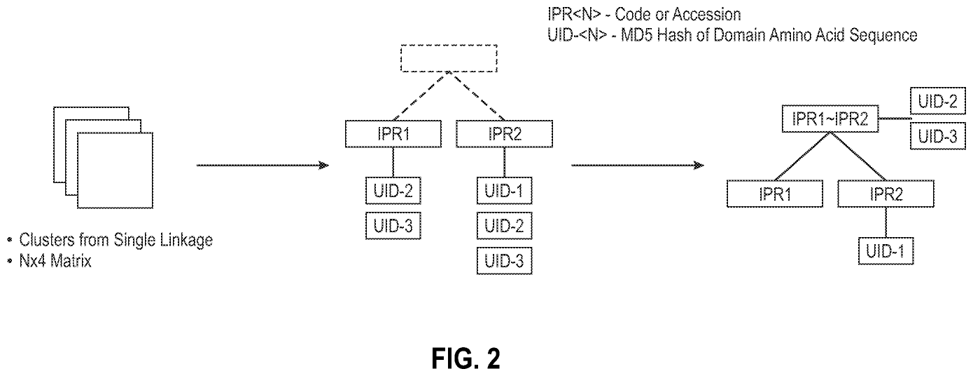

FIG. 2 shows a representative, but non-limiting, a binary tree generated from the theoretical clustering. Within the binary tree system, two leaf nodes are initialized using code accession as the node names and related domain UIDs as the node data. An MD5 hash may be used to test the integrity of the UIDs. Internal nodes are constructed by the concatenation of the left and right IPRs where the IPR child node names become the final node names. The intersection of the domain UIDs become the node data, which represents the lowest common ancestor (LCA). The process shown in FIG. 2 is repeated until the tree arrives at the root.

In another embodiment, the classification tool is a k-mer based classifier. Two examples of k-mer based classifiers are PRROMenade (International Business Machines Corporation, Armonk, N.Y., USA) and Kraken™2 (LGC Biosearch Technologies, Middlesex, UK). PRROMenade is a microbiome classification tool that uses variable length k-mers for coding systems that are already organized as a tree. Kraken2 is a taxonomic classification system that matches each k-mer within a query sequence to the LCA of all genomes containing the exact k-mer. The clustering of the sequences within the functional classifier result in the k-mer classification tool being capable of identifying sequences within the taxonomic tree for the microbiome classification.

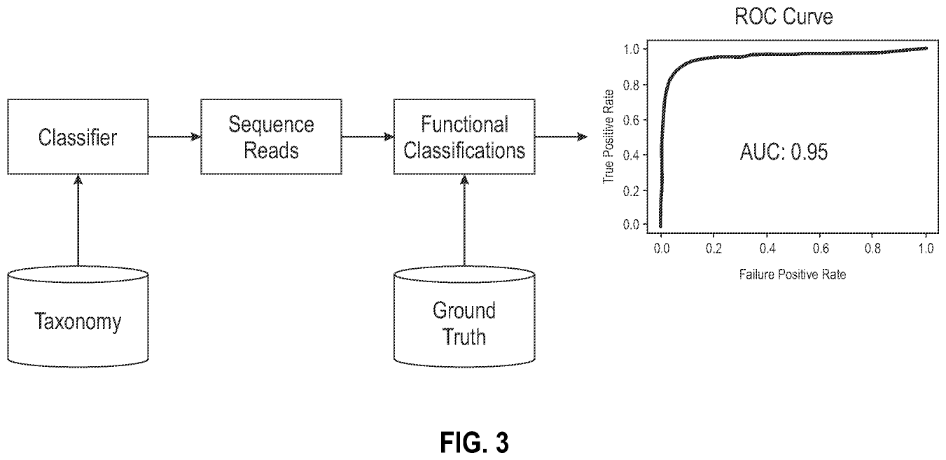

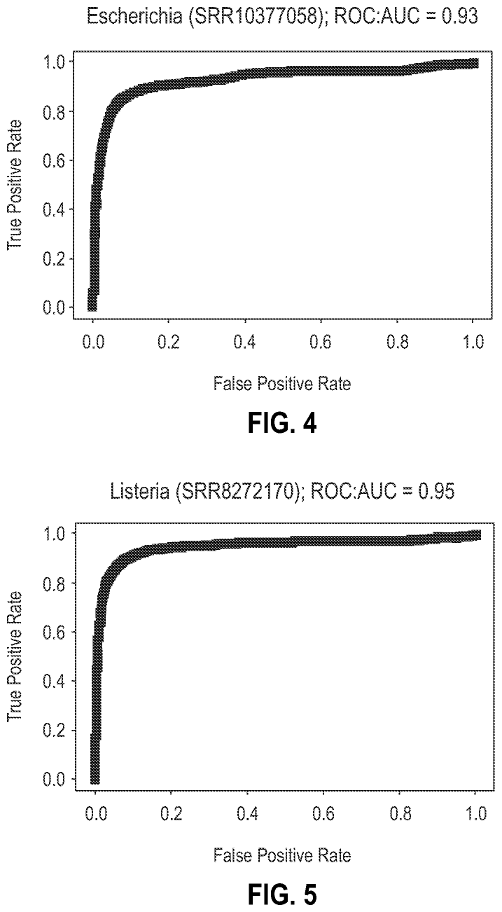

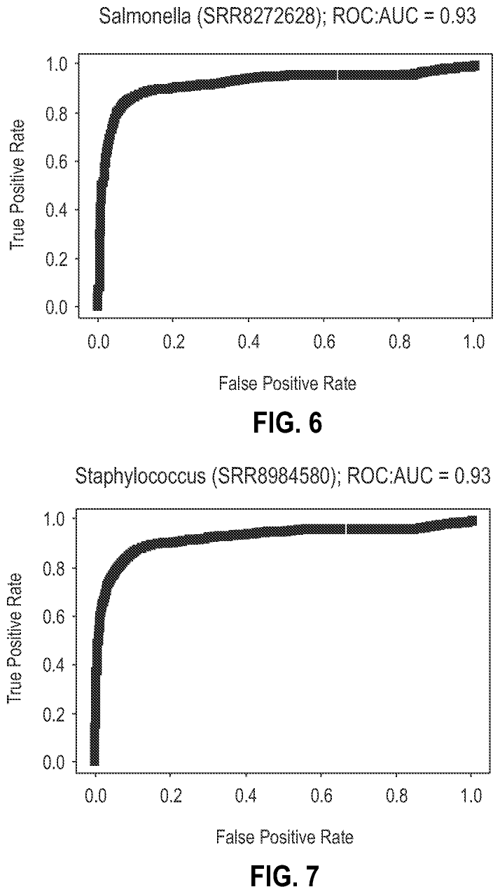

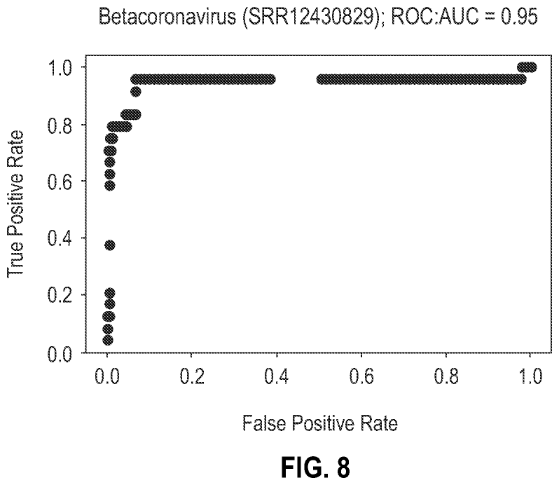

With reference to FIG. 3, the performance of the classifier is quantified by analysis of raw unassembled reads from whole genome sequence (WGS) data that have been independently annotated for function or bioactivity. From the WGS ground truth data, ROC curves and AUC are measured to quantify classifier performance and to choose the best strategy for the classifier construction. FIGS. 4-8 show application of the classifier to classify four bacterial species (Escherichia coli, FIG. 4), Listeria monocytogenes, FIG. 5), Salmonella enterica, FIG. 6), and Staphylococcus aureus, FIG. 7), and one RNA virus (Betacoronavirus, FIG. 8). In each classification, the AUC:ROC is 0.93 or 0.95 representing a high accuracy rate for the functional classifier.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, graphics processing units (GPU), field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be accomplished as one step, executed concurrently, substantially concurrently, in a partially or wholly temporally overlapping manner, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various aspects and/or embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the aspects and/or embodiments, the practical application or technical improvement over technologies found in the marketplace, or to enable others of ordinary skill in the art to understand the aspects and/or embodiments disclosed herein.

### Experimental

The following examples are set forth to provide those of ordinary skill in the art with a complete disclosure of how to make and use the aspects and embodiments of the invention as set forth herein. While efforts have been made to ensure accuracy with respect to variables such as amounts, temperature, etc., experimental error and deviations should be considered. Unless indicated otherwise, parts are parts by weight, temperature is degrees centigrade, and pressure is at or near atmospheric. All components were obtained commercially unless otherwise indicated.

### EXAMPLE 1

For microbiome testing, a classifier was prepared according to the steps of FIG. 1 using InterProScan as the coding system and FGP (Functional Genomics Platform) as the reference database and a cosine function for the matrix pair-wise distance measurements. Following establishment of the classifier, the following three synthetic microbiome datasets were constructed: 1. a DNA complex; 2. a DNA Human Gut; and 3. an RNA Human Gut. The three microbiome test sets were classified with the classifier and the performance of the classification was measured using AUC:ROC. Ground truth was created for each test using InterProScan annotations obtained from the FGP and the InterProScan website.

FIGS. 4-7 show the AUC:ROC results for the following four bacterial species represented within the Human Gut synthetic microbiome datasets (MCT=minimum cutoff threshold or operating point): Escherichia coli (FIG. 4; AUC=0.93), Listeria monocytogenes (FIG. 5; AUC:ROC=0.95), Salmonella enterica (FIG. 6; AUC:ROC=0.95), and Staphylococcus aureus (FIG. 7; AUC:ROC=0.93). FIG. 8 shows the AUC:ROC results for the viral RNA Betacoronavirus (AUC:ROC=0.95).

We claim:

1. A method of constructing a microbiome classifier comprising:
selecting a reference database comprising a set of biological sequences, wherein each biological sequence is annotated with a code from at least one coding system;
constructing a matrix comprising rows, columns, and cells, wherein each row represents one code from the at least one coding system, each column represents a single biological sequence from the set, and the cells show the presence, absence, or frequency of the single biological sequences for one or more codes of the at least one coding system;
computing pair-wise distance between the rows of the matrix to arrive at a single pair-wise distance value for each code in the set, wherein the pair-wise distance computations between all of the rows of the matrix is an N×N matrix, wherein N is the number of codes in the matrix;
clustering the pair-wise distance values for each code to form a data structure tree comprising clusters, wherein the clusters represent a relationship between a code of the at least one coding system and one or more biological sequences;
constructing a taxonomic tree comprising internal nodes and leaf nodes wherein the internal nodes represent the clusters of the data structure tree and the internal nodes and the leaf nodes represent the biological sequences; and
applying a classification tool to the final taxonomic tree to classify a microbiome comprised of the biological sequences.

2. The method of claim 1, wherein the biological sequences are selected from the group consisting of a pair-end read, a gene sequence, a protein sequence, and combinations thereof.

3. The method of claim 1, wherein the at least one coding system annotates the biological sequences with functional codes.

4. The method of claim 3, wherein the functional codes are selected from the group consisting of nucleic and/or amino acid pathways, chemical reactions involving nucleic acid and/or proteins, protein reactions initiated by enzymes, hierarchical functional codes, and combinations thereof.

5. The method of claim 1, wherein the at least one coding system relates microbial function to a sequence selected from the group consisting of a gene, a protein, a motif, a domain, and combinations thereof.

6. The method of claim 1, wherein the at least one coding system is hierarchical or non-hierarchical.

7. The method of claim 1, wherein each biological sequence in the columns of the matrix is coded with a single unique identifier (UID).

8. The method of claim 7, wherein the UID represents a unique sequence selected from the group consisting of a gene, a protein, a motif, a domain, and combinations thereof.

9. The method of claim 1, wherein the matrix is a sparse matrix and each row of the matrix is a sparse vectorization of the biological sequences that are related to one or more codes of the at least one coding system.

10. The method of claim 1, wherein the pair-wise distance between the rows of the matrix is calculated with a metric selected from the group consisting of cosine similarity, Euclidean distance, Hamming distance, Jaccard distance, and combinations thereof.

11. The method of claim 1, wherein the classification tool is a k-mer based classifier.

12. A method of constructing a microbiome classifier comprising:
selecting a reference database comprising a set of protein domain sequences, wherein each protein domain sequence is annotated with a code from at least one coding system;
constructing a matrix comprising rows, columns, and cells, wherein each row represents one code from the at least one coding system, each column represents a single protein domain sequence from the set, and the cells show the presence, absence, or frequency of the single protein domain sequences for one or more codes of the at least one coding system;
computing pair-wise distance between the rows of the matrix to arrive at a single pair-wise distance value for each code in the set, wherein the pair-wise distance computations between all of the rows of the matrix is an N×N matrix, wherein N is the number of codes in the matrix;
clustering the pair-wise distance values for each protein domain sequence to form a data structure tree comprising clusters, wherein the clusters represent a relationship between one or more codes of the at least one coding system and one or more protein domain sequences;
constructing a taxonomic tree comprising internal nodes and leaf nodes wherein the internal nodes represent the clusters of the data structure tree and both the internal nodes and the leaf nodes represent the protein domain sequences; and
applying a classification tool to the final taxonomic tree to classify a microbiome comprised of the protein domain sequences.

13. The method of claim 12, wherein the protein domain sequence is annotated with functional information relating to (i) enzymes that catalyze reactions with the protein domain sequence and/or (ii) reactions and/or pathways that the protein domain sequence undergoes.

14. The method of claim 12, wherein the at least one coding system relates microbial function to domain sequence phenotype.

15. The method of claim 12, wherein the at least one coding system is hierarchical or non-hierarchical.

16. The method of claim 12, wherein each protein domain sequence in the columns of the matrix is coded with a single unique identifier (UID).

17. The method of claim 16, wherein each UID represents a unique protein domain sequence.

18. The method of claim 12, wherein the matrix is a sparse matrix and each row of the matrix is a sparse vectorization of the protein domain sequences that are related to one or more codes of the at least one coding system.

19. The method of claim 12, wherein the pair-wise distance between the rows of the matrix is calculated with a metric selected from the group consisting of cosine similarity, Euclidean distance, Hamming distance, Jaccard distance, and combinations thereof.

20. The method of claim 12, wherein the classification tool is a k-mer based classifier.
